(19) United States
(12) Patent Application Publication
(10) Pub. No.: US 2023/0340705 A1
(43) Pub. Date: Oct. 26, 2023

(54) HEMP PAD AND RELATED METHOD OF MANUFACTURE

(71) Applicant: LYALL DONALD BATES, GILBERT PLAINS (CA)

(72) Inventor: LYALL DONALD BATES, GILBERT PLAINS (CA)

(21) Appl. No.: 18/298,438

(22) Filed: Apr. 11, 2023

Related U.S. Application Data
(60) Provisional application No. 63/333,732, filed on Apr. 22, 2022.

(51) Int. Cl. D03D 15/217; D01C 1/02

(52) U.S. Cl. CPC D03D 15/217; D01C 1/02

(57) ABSTRACT

A hemp pad comprises first and second opposite exterior faces; a periphery interconnecting the exterior faces; and a body defining the first and second exterior faces and essentially comprising bast fibre. Bast fibre comprises a plurality of fibril bundles, each bundle including fibrils and polymeric binder material holding the fibrils together. The polymeric binder material comprises at least one of lignin, pectin, hemicellulose and cellulose. The fibrils of each bundle are separated from each other along portions of lengths thereof. The fibril bundles are joined together to form the body by cohesion of the polymeric binder material. Opening within individual fibril bundles and attachment of different bundles to one another to form a body of hemp fibre is a result of heat treatment to degrade the polymeric binder material. A related method for processing hemp for use as an absorption pad is also disclosed.

This application claims the benefit under 35 U.S.C. 119(e) of U.S. Provisional Application Ser. No. 63/333,732 filed Apr. 22, 2022.

### FIELD OF THE INVENTION

The present invention relates to a hemp pad and related method of manufacture, and more particularly to a hemp pad in which constituent hemp fibre is heat treated.

### BACKGROUND

It is generally known to make mats or pads from industrial hemp for absorbing fluids, that is liquids and gases. Hemp comprises shive and bast fibre as constituent components thereof.

### SUMMARY OF THE INVENTION

According to an aspect of the invention there is provided a hemp pad comprising:
first and second opposite exterior faces spaced apart by a thickness of the hemp pad;
a periphery interconnecting the first and second exterior faces;
a body defining the first and second exterior faces, wherein the body essentially comprises bast fibre;
wherein the bast fibre comprises a plurality of fibril bundles, wherein each of the fibril bundles includes a plurality of fibrils and polymeric binder material holding the fibrils together, wherein the polymeric binder material comprises at least one of lignin, pectin, hemicellulose and cellulose;
wherein, in each fibril bundle, the fibrils thereof are separated from each other along portions of lengths thereof; and
wherein the fibril bundles are joined together to form the body by cohesion of the polymeric binder material.

This provides an arrangement of hemp pad with improved performance for absorbing fluids as compared to commercially available products.

Preferably, the fibrils of the bundles are interwoven.

Preferably, the bast fibre is heat treated in air.

Preferably, the bast fibre is heat treated in air at a temperature in a range from about 280 degrees Celsius to about 350 degrees Celsius.

In one arrangement, a proportion of the polymeric binder material in the body comprises about 40% to about 45% by mass.

In one arrangement, the thickness is about 3/16 inches to about 9/16 inches.

In one arrangement, the hemp pad is used to absorb fluids.

In one arrangement, the hemp pad is used to mechanically remove debris from an article.

According to another aspect of the invention there is provided a method for processing hemp for use as an absorption pad, comprising:
providing bast fibre of the hemp, wherein the bast fibre comprises a plurality of fibril bundles, wherein each of the fibril bundles includes a plurality of fibrils and polymeric binder material holding the fibrils together, wherein the polymeric binder material comprises at least one of lignin, pectin, hemicellulose and cellulose;
degrading the polymeric binder material by applying heat to the bast fibre, so that the fibril bundles are opened; and
collecting the opened fibril bundles in a container such that the opened fibril bundles are joined to each other by the polymeric binder material thereof upon cooling to form a body of hemp fibre.

This provides an arrangement of hemp processing method which yields hemp fibre in a format having improved performance for absorbing fluids as compared to commercially available products.

Preferably, the heat is applied at a temperature in a range from about 280 degrees Celsius to about 350 degrees Celsius.

Preferably, the method further includes interweaving the fibrils of the opened fibril bundles.

In one arrangement, interweaving the fibrils is performed while the body of hemp fibre is warmer than ambient temperature. Preferably, a temperature of the body of hemp fibre for interweaving is about 100 degrees Fahrenheit to about 125 degrees Fahrenheit.

Preferably, the method further includes compacting the body of hemp fibre.

Preferably, when the method includes interweaving the fibrils of the opened fibril bundles, compacting the body of hemp fibre is performed after interweaving the fibrils.

In one arrangement, compacting the body of hemp fibre comprises roller compaction.

### BRIEF DESCRIPTION OF THE DRAWINGS

The invention will now be described in conjunction with the accompanying drawings in which:

FIG. 1 schematically shows a side view of an arrangement of hemp pad according to the present invention;

FIG. 2 schematically shows a plan view of the arrangement of hemp pad of FIG. 1 with some features omitted for convenience and clarity of illustration;

FIG. 3 is a flowchart of steps of an arrangement of method processing hemp for use as an absorption pad according to the present invention; and

FIG. 4 is a schematic diagram of a system for implementing the arrangement of method represented by FIG. 3.

In the drawings like characters of reference indicate corresponding parts in the different figures.

### DETAILED DESCRIPTION

Referring to the accompanying drawings, there is shown a hemp pad 10 which is particularly but not exclusively suited for use to absorb fluids in either liquid or gaseous form.

The hemp pad 10 generally comprises first and second opposite exterior faces 12, 13, which are spaced apart by a thickness T of the hemp pad, and a periphery 15 interconnecting the opposite exterior faces 12, 13. In the illustrated arrangement, the first and second faces 12, 13 and periphery 15 collectively define a whole exterior of the pad 10. Depending on the thickness T of the pad, the periphery 15 may be a peripheral edge or a peripheral surface which in either case encompasses the first and second faces 12, 13 which are primary faces collectively forming a majority portion of a total surface area of the pad. The exterior faces 12, 13 are for respectively engaging items, for example foodstuff. The exterior faces 12, 13 are uncovered and detached from any other material so as to be exposed to and available for contacting items such as foodstuff.

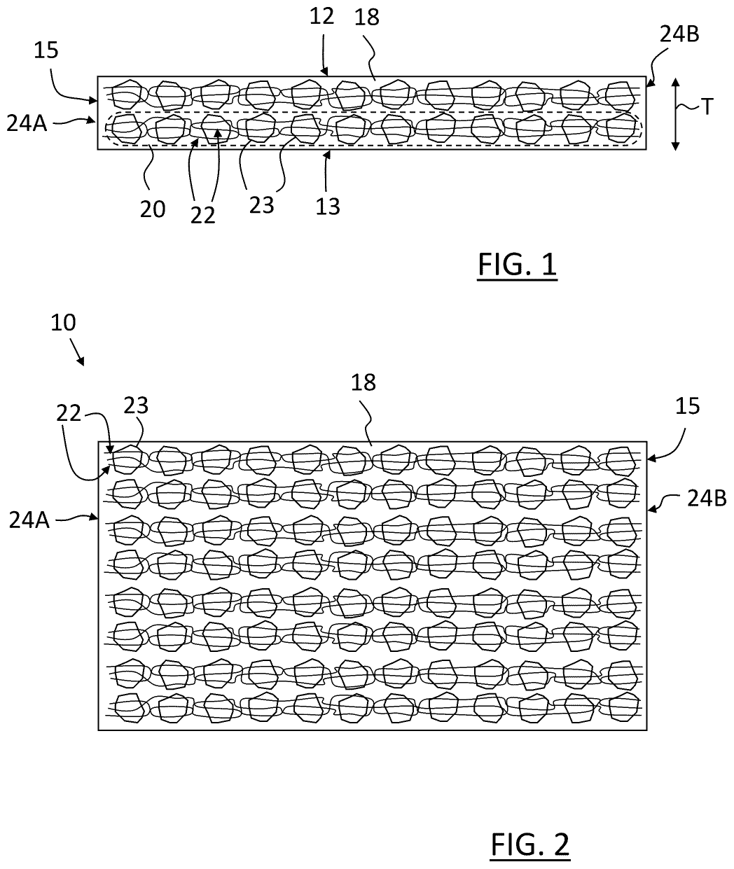

Furthermore, the hemp pad 10 includes a body 18 defining the first and second exterior faces 12, 13. The body 18 essentially comprises bast fibre. That is, the body 18 of the pad 10 is made up of bast fibre only without any foreign or non-hemp material or additives. It will be appreciated that FIGS. 1 and 2 show smooth and planar exterior faces 12, 13 and planar peripheral surfaces at the periphery 15 for convenience of illustration; however, since the bast fibre is irregular in shape, contours of these exterior faces are irregular.

Generally speaking, bast fibre of hemp comprises a plurality of fibril bundles 20, and each fibril bundle 20 includes a plurality of fibrils 22 and polymeric binder material 23 holding the fibrils together. The polymeric binder material 23 comprises at least one of lignin, pectin, hemicellulose and cellulose. That is, the polymeric binder material is naturally occurring to the hemp.

In the illustrated arrangement, the pad body 18 is shown as comprising 14 fibril bundles arranged in two layers (more clearly shown in FIG. 1) between the primary exterior faces 12, 13, with seven fibril bundles in each layer (more clearly shown in FIG. 2) each extending longitudinally a full length of the body between opposite ends 24A, 24B.

The bast fibre of the body 18 is processed so that the fibrils 22 of each fibril bundle 20 are separated from each other along portions of lengths thereof. In other words, the fibrils of each bundle are spaced from each other in crosswise directions to their lengths, at least along one or more lengthwise portions or sections of the fibrils. This separation may take place where there is no polymeric binder material interconnecting the fibrils of a common bundle or along lengthwise sections of the fibril bundle where the natural polymeric binder material has degraded or deteriorated from its initial condition prior to any processing to chemically alter a condition of the bast fibre. Despite separation or divergence of individuals ones of the fibrils from each other in a common bundle, the fibril bundles 20 are (still) substantially intact, meaning that the fibril bundles contain the same number of fibrils as in their initial pre-processing condition. Thus are formed air pockets or cavities 26 in the body 18 which can trap fluids, such that the pad acts to absorb same.

More specifically, this processing to open the fibril bundles, that is to separate the fibrils of a common bundle from each other in a direction crosswise to their lengths, comprises heat treatment, that is the application of heat to (or heating of) the bast fibre, to degrade the natural polymeric binder material so as to reduce an amount and/or quality of this material. Thus, after heat treatment, a remaining amount and/or quality of the polymeric binder material is less than an initial amount and/or quality thereof naturally occurring to the bast fibre and substantially intact therewith once shive of the hemp has been separated.

Since there is a remaining amount of polymeric binder material after heating, the fibril bundles 20 are joined together to form the body 18 by cohesion of the remaining polymeric binder material 23. The remaining polymeric binder material of a respective bundle may bind to fibrils of other bundles or to polymeric binder material thereof.

Thus, the body 18 of the pad 10 is substantially homogenous and basically comprises an array of fibril bundles, typically three dimensional, with substantially uniform density and distribution of the treated bast fibre. The body is free of any backing or substrate, which is typically denser than fibres connected thereto and forming an outer or exterior surface of such a body.

To reinforce the pad body 18, the fibrils 22 of the bundles 20 are interwoven. Freed or separated lengths of the fibrils which are sufficiently loose are available to be interwoven, and are intertwined by passing the pad body 18 through a weaving machine, such as a needlepunch, configured to interweave fibres of a material input thereto.

In the illustrated arrangement, the bast fibre is heat treated in air and at a temperature in a range from about 280 degrees Celsius to about 350 degrees Celsius. In this temperature range, the hemp polymeric binder material is softened and partially destroyed or deteriorated to loosen individual fibrils of the bundles while the bundles remain substantially intact.

Consequently, a proportion of the polymeric binder material in the body of the pad comprises about 40% to about 45% by mass. This is less than the initial proportion of the polymeric binder material intact with the fibril bundles after separation from shive and before further processing. The proportion of polymeric binder material before separation is about 57% by mass.

In the illustrated arrangement, the thickness of the hemp pad is about 3/16 inches to about 9/16 inches. Thus the hemp pad is relatively thin so that when inserted into a container an available volume thereof is minimally and preferably negligibly reduced.

This provides an arrangement of hemp pad with improved performance for absorbing fluids as compared to commercially available products.

There is also disclosed a method for processing hemp for use as an absorption pad. Hemp comprises shive and bast fibre as constituent components thereof.

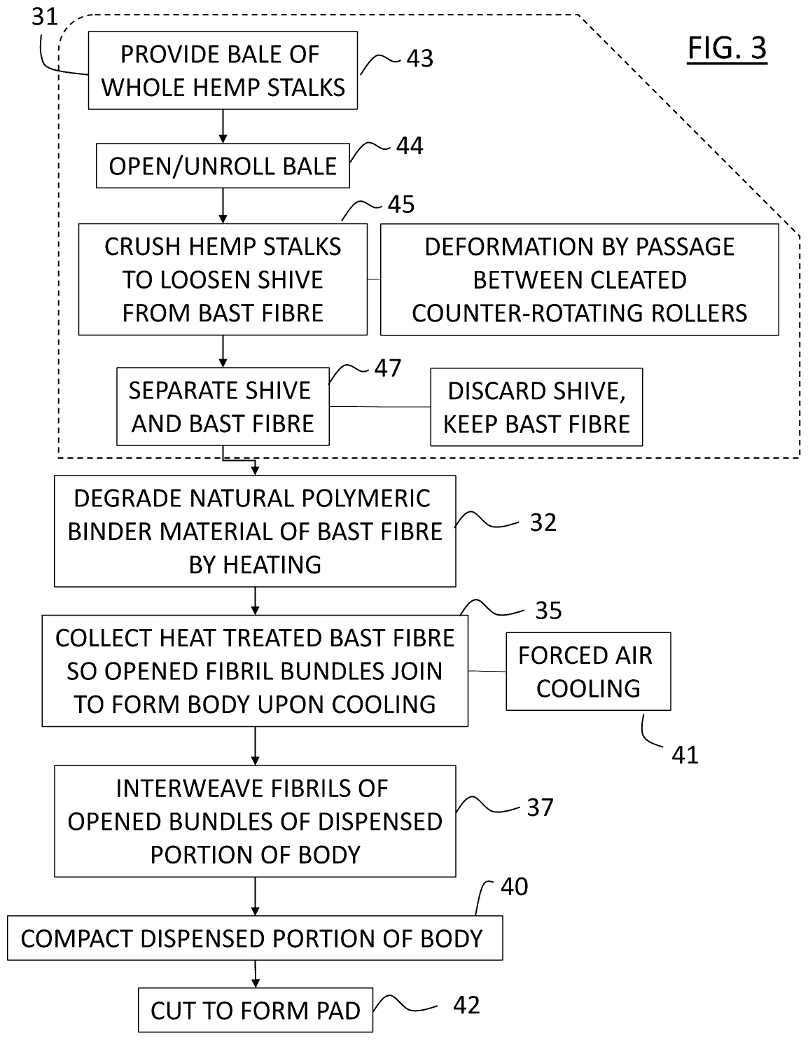

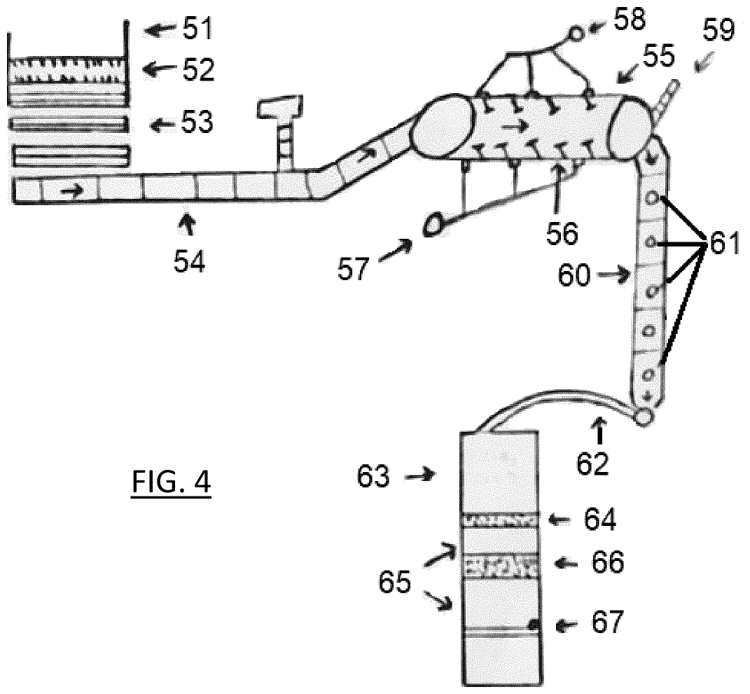

With reference to FIGS. 3 and 4, the method generally comprises the steps of:
providing bast fibre of the hemp, as represented by a group of steps indicated at 31;
as indicated at step 32, degrading the polymeric binder material 23 by applying heat to the bast fibre so that the fibril bundles 20 are opened; and
collecting the opened fibril bundles in a container such that the opened fibril bundles are joined to each other by the polymeric binder material thereof upon cooling to form a body of hemp fibre, as represented by step 35.

When the natural polymeric binder material is degraded at step 32, an amount and/or quality of this binder material is reduced. Thus are formed, from the bast fibre having an initial condition, with polymeric binder material of an initial condition, opened fibril bundles with polymeric binder material thereof having a degraded or deteriorated condition either in amount and/or quality. In other words, in this manner are formed the opened fibril bundles having remaining polymeric binder material of a reduced amount and/or quality than the bast fibre in the initial condition.

At step 35, the opened, heat-treated fibril bundles are collected while still warm after heat treatment such that the polymeric binder material is sufficiently soft to be adherable. Thus, the collection step is performed while the opened fibril bundles is in a heated condition in which the polymeric binder material is soft for cohesion.

In the illustrated arrangement of processing method, the heat is applied at a temperature in a range from about 280 degrees Celsius to about 350 degrees Celsius.

Additionally, the method preferably includes interweaving the fibrils of the opened fibril bundles, as indicated at step 37. In the illustrated arrangement, the interweaving step is performed while the body of hemp fibre is warmer than ambient temperature. For the interweaving step, the body of hemp fibre is preferably at a temperature of about 100 degrees Fahrenheit to about 125° F.

Furthermore, the method preferably further includes compacting the body of hemp fibre, as indicated at step 40. In the illustrated arrangement, compacting the body of hemp fibre comprises roller compaction. Also, the compacting step 40 is performed after interweaving the fibrils at step 37. Compressive forces of the compaction step are exerted in directions substantially normal to primary faces of the body of hemp fibre.

Since the opened fibril bundles join to form the body of hemp fibre upon cooling after the heat treatment or application step, the method includes a cooling step at 41 to expedite formation of the fibrous body for further processing. In the illustrated arrangement, the cooling step 41 comprises forced air cooling and is performed concurrently with the collection or (temporary) storage step at 37. The cool air, at a lower temperature than the applied heat of the heat treatment step, and preferably at about 75° F., is blown into the container which temporarily houses the opened fibril bundles for cohesion in the container.

The substantially unitary fibrous body formed in the container is gradually dispensed for further processing, which includes the compaction step 40 and cutting to a desired size and shape of pad thereafter, at step 42.

To obtain the bast fibre for processing to form the hemp pad, as represented at 31, this typically involves a series of steps including:
providing a bale of whole hemp stalks, as indicated at step 43, which have been harvested from a growing field and which are seedless because seeds have been removed in the field;
opening or unrolling the bale, as indicated at step 44, to dispense harvested whole hemp stalks for further processing;
crushing the whole hemp stalks to loosen the shive from the bast fibre, as indicated at step 45, which in the illustrated arrangement is achieved by passing the dispensed portion of the bale through counter-rotating cleated rollers to deform the whole stalks; and
separating the shive and the bast fibre, as indicated at step 47, which in the illustrated arrangement comprises agitation of the crushed whole hemp stalks and screening the crushed whole hemp stalks to mechanically separate the shive and the bast fibre.

The hemp stalks are considered whole in that they comprise both shive and bast fibre which are intact.

In the illustrated arrangement, the shive is discarded and the bast fibre is kept for further processing.

This provides an arrangement of hemp processing method which yields hemp fibre in a format having improved performance for absorbing fluids as compared to commercially available products.

The hemp pad as described hereinbefore may be used to absorb fluids or to mechanically remove debris from an article, especially in conjunction with washing with water and a soap or detergent.

With reference to FIG. 4, an example of system for performing the method to process hemp for use as an absorption pad includes the following: a bale unroller 51 which has a table on which the bale is placed. The table is controlled by an electrical motor with variable speed control; when the table is raised, the bale is located or positioned in engagement with rollers 52 with spikes or pointed tips, which rollers rotate with variable speed to evenly peel off the hemp stalks from the bale. Then, the hemp stalks pass through plural sets of rollers 53, in this case three pairs, which are crimper rollers with cleats to separate the shive from the fibre. Then, the shive and fibre are transferred by a conveyor 54 and sent through a screener 55. The screener 55 has a screen size of 1.5 inches and the screen has rubber paddles 56 inside the screener to stir the fibre around to help separate the shive through the screen 55. A blower fan blows air into the bottom of the screener at 57 and a fan collecting the air from the top of the screener at 58 removes the dust and small partials from the fibre. The shive that is screened is removed from the bottom of the screener with a conveyor 59. Then, the clean or separated fibre from the screener is dropped onto a conveyer 60 that is heated with heat elements 61 to soften the lignin which leads to opening of the fibre into individual fibers, releasing the lignin from the fibre. Then, the hot individual fibres are blown by air at 62 to the holding tank 63, where the holding tank has cool air be blown into the tank, as the fibres are fed out of the bottom of the holding tank 63 evenly by a set of rollers with small indents on the roller 64 then move by a conveyor 65 to the needle punch machine 66 where the warm fibers are cross over each other with a needle punch. Then the mat is formed and moved by conveyor 65 to the circular fiber disc blade 67 to be cut into any size as desired.

Prior to processing, during harvesting of the hemp, seed thereof is removed from the hemp stalks by a harvester and then the hemp stalks are cut in the field and left in the field to dry down to a prescribed initial moisture content of 13% or less by weight. After drying to the prescribed initial moisture content, the seedless whole stalks are baled and stored.

Steps in producing the hemp mats comprise the following:
1. The bale is unrolled by a bale opener which has a table to set the bale on to start with, then the table is controlled by an electrical cylinder which controls the speed and force to raise the table which then forces the bale on the table to the rollers with spikes. The amount or thickness of hemp stalks unrolled is controlled by a roller with spikes which can be set from one-half to four inches. For example, if the spikes are set to one inch projecting from an outer rounded surface of the roller, then the thickness of hemp stalks unrolled from the bale is one inch.
2. After dispensing the whole hemp stalks from the bale, the whole stalks are put through three sets of crimper rollers with the bottom rollers rotating clockwise and the top rollers rotating anticlockwise. All of these rollers have axially-extending cleats arranged in angularly spaced relation to one another. Opposite cooperating pairs of the crimper rolls are spaced apart so as to crush the hemp stalks by deformation. The rollers which are precision timed and as the hemp stalks pass through the rollers these high-pressured rollers crimped every other inch to break apart the shive from the base fibre.
3. After passing through the crimper rollers, the fibers and the shive are put through a screener, for example a trommel screener.
4. The trommel screener has a screen size of 1.5 inches to let the shive fall through the screen to the bottom where the shives are removed by a conveyor. The trommel screener is enclosed with a cover around the screener. Air is forced from the bottom of the screener with a plurality of inlet holes, in this case four, and air is collected from the top of the screener with a plurality of outlet holes, preferably of a common quantity as the number of inlet holes, in this case four. This apparatus acts to collect the fine particles from the bast fibre. The trommel screener has rubber paddles inside the screener to stir the hemp fibers around to help remove the shive.
5. After screening, and the shive has been removed from the bast fibre, the bast fibre is transferred to a belt conveyor with a perforated upper support surface formed by opposite runs of the conveyor, formed by, for example, a steel perforated chain. Radiant heat elements or forced electrical heat elements are arranged on above and below the belt conveyer with an adjustable thermostat to control the amount of heat in which is set between about 280° C. and about 350° C. depending on an moisture content of the bast fibre before entering into the heated conveyor. Heating the fibers between about 280° C. and about 350° C. results in softening of the natural polymeric binder material such as lignin, hemicellulose and cellulose leading to opening of fibril bundles of the bast fibre into individual fibres or fibrils. Due to the heat treatment, between about 20% to about % of the fibril bundles has substantially no or less lignin or hemicellulose or cellulose which permits absorption of a higher amount of gases and moisture.
6. The warm fibril bundles are then blown to the holding tank, and cool air is blown into the tank to cool the fibril bundles with reduced polymeric binder material thereon. The binder material of different bundles attaches to each other and to the fibrils thereof. However, there is in general less natural binder material present in the treated bast fibre leaving a larger proportion of a volume of the body of hemp fibre as void or empty in the form of pockets. After cooling, the treated fibre is dispensed or removed from the holding bin by a roller with small indents thereon and transferred to the needle punch where individual fibrils are crossed over each other by the needle punch machine. Afterwards, the woven body passes through high pressure smooth rollers to compact the body.
7. Then the pad is cut with a circular fiber disc blade to a desired size.
8. The hemp fibre pad or mat is suited for use to absorb moisture and gases that are given off from fresh or perishable produce such as vegetables and fruits which slows down ripening and spoilage of the vegetables and fruits up to 20 days.
9. The pad can be used during shipping of fresh produce by setting or lining one or more pads in a shipping container to receive the fresh produce. The mat can be laid or positioned on a support structure to carry such foodstuff, for example fruit bowls, in refrigerators, in Grocery stores or Supermarkets.
10. This invention is where the hemp fibers being cleaned and then heated to separate the lignin/glues from the fibers and still have the lignin/glue and fiber together which will form the hemp pad then allows the high amount of absorbing to take place when these mats are used for absorbing moisture and gases.
11. Coconut fiber also known as Coir can also be used in process, or cotton or Coconut Coir could be blended with hemp to make food preserving pads. This patent is about how the hemp stalks are unrolled from the bales and how to control the amount being unrolled at one time.

As described hereinbefore, the present invention relates to using heat to soften and to damage or destroy some but not all-natural polymeric binder material of hemp bast fibre to open fibril bundles of the bast fibre to yield a more porous body of fibre with ability to absorb and potentially trap more gas and moisture and trap them inside the porous structure of the fibers, while leaving some natural polymeric binder material for holding the fibril bundles of the pad body together.

Hemp pads or mats have a dynamic role in extending shelf life or improving safety or sensory properties while maintaining the quality of the food, advances in many areas, including delayed oxidation and controlled respiration rates, microbial growth, and moisture migration.

The presence of oxygen in packaged foods causes many deteriorative reactions such as nutrient losses, color changes, off-flavor development, and microbial growth, and it also has a considerable effect on the respiration rate and ethylene production in fruits and vegetables. Oxygen-sensitive food which has been packaged using hemp pads have proven to absorb the gases and moisture and keep the vegetables and fruits fresh for a longer period of time.

There are no chemicals or binders added to the hemp, so the hemp pads are 100% natural.

The hemp pad is made from 100% hemp fibre. There are no additives. The hemp pad is all natural, biodegradable and compostable.

A preferred use for the hemp pad as a food preserving pad. Other uses include absorbing gases, moisture and odors, that is, in general, to absorb fluids.

The hemp pad is usable in fridges, counter tops, fruit bowls, lunch pails, coolers, grocery stores and supermarkets laid underneath the fruits and vegetables. shipping fruits and Vegetables where it can be set inside on the bottom and on the sides of the boxes or creates to keep the fruits and Vegetables fresh while in transport.

The hemp pad can be used underneath pet cages to absorb odors, can be used on cement floors to absorb oil spills, can be used for chemical spill to absorb the chemical, can be used in green houses to absorb gases, can be used in flower pots to collect moister and release moister back into the soil and will biodegradable in to the soil over time and add natural nitrogen to the soil, control weed growth in the plant pots

The pad can be washed in warm water to remove the gases, moisture and odors and dried and reused again.

Also, the pad can be used for a wash pad for cleaning dishes and pots and pans and can be used to wash stove tops etc. and will not scratch stove tops.

The scope of the claims should not be limited by the preferred embodiments set forth in the examples but should be given the broadest interpretation consistent with the specification as a whole.

1. A hemp pad comprising:
first and second opposite exterior faces spaced apart by a thickness of the hemp pad;
a periphery interconnecting the first and second exterior faces;
a body defining the first and second exterior faces, wherein the body essentially comprises bast fibre;
wherein the bast fibre comprises a plurality of fibril bundles, wherein each of the fibril bundles includes a plurality of fibrils and polymeric binder material holding the fibrils together, wherein the polymeric binder material comprises at least one of lignin, pectin, hemicellulose and cellulose;
wherein, in each fibril bundle, the fibrils thereof are separated from each other along portions of lengths thereof; and
wherein the fibril bundles are joined together to form the body by cohesion of the polymeric binder material.

2. The hemp pad of claim 1 wherein the fibrils of the bundles are interwoven.

3. The hemp pad of claim 1 wherein the bast fibre is heat treated in air.

4. The hemp pad of claim 3 wherein the bast fibre is heat treated in air at a temperature in a range from about 280 degrees Celsius to about 350 degrees Celsius.

5. The hemp pad of claim 3 wherein a proportion of the polymeric binder material in the body comprises about 40% to about 45% by mass.

6. The hemp pad of claim 1 wherein the thickness is about 3/16 inches to about 9/16 inches.

7. Use of the hemp pad of claim 1 to absorb fluids.

8. Use of the hemp pad of claim 1 to mechanically remove debris from an article.

9. A method for processing hemp for use as an absorption pad, comprising:
providing bast fibre of the hemp, wherein the bast fibre comprises a plurality of fibril bundles, wherein each of the fibril bundles includes a plurality of fibrils and polymeric binder material holding the fibrils together, wherein the polymeric binder material comprises at least one of lignin, pectin, hemicellulose and cellulose;
degrading the polymeric binder material by applying heat to the bast fibre, so that the fibril bundles are opened; and
collecting the opened fibril bundles in a container such that the opened fibril bundles are joined to each other by the polymeric binder material thereof upon cooling to form a body of hemp fibre.

10. The method of claim 9 wherein the heat is applied at a temperature in a range from about 280 degrees Celsius to about 350 degrees Celsius.

11. The method of claim 9 further including interweaving the fibrils of the opened fibril bundles.

12. The method of claim 11 wherein interweaving the fibrils is performed while the body of hemp fibre is warmer than ambient temperature.

13. The method of claim 9 further including compacting the body of hemp fibre.

14. The method of claim 13 wherein, when the method includes interweaving the fibrils of the opened fibril bundles, compacting the body of hemp fibre is performed after interweaving the fibrils.

15. The method of claim 14 wherein compacting the body of hemp fibre comprises roller compaction.
